(19) United States
(12) Patent Application Publication
(10) Pub. No.: US 2012/0115174 A1
(43) Pub. Date: May 10, 2012

(54) URINARY TRYPSIN INHIBITORS AS DIAGNOSTIC AID FOR INTERSTITIAL CYSTITIS

(75) Inventor: Michael J. Pugia, Granger, IN (US)

(73) Assignee: SIEMENS HEALTHCARE DIAGNOSTICS INC., Tarrytown, NY (US)

(21) Appl. No.: 13/384,013

(22) PCT Filed: Jul. 12, 2010
(86) PCT No.: PCT/US2010/041678

§ 371 Date: Jan. 13, 2012

Related U.S. Application Data
(60) Provisional application No. 61/226,359, filed on Jul. 17, 2009.

(51) Int. Cl. G01N 33/569; C12Q 1/04; C12M 1/34

(52) U.S. Cl. 435/7.92; 435/287.1; 435/34; 435/7.1

(57) ABSTRACT

A method aiding the diagnosis of interstitial cystitis involving the combination of an infection marker and an inflammation marker. More specifically, the method includes correlating the presence of urinary trypsin inhibitors in urine with the absence of traditional infection markers in urine to aid in the diagnosis of interstitial cystitis. The method provides for a differential diagnosis between kidney disease, infection and chronic inflammation with a noninvasive urine test. Assay devices and kits, as well as analyzers and systems are also described that utilize the methodology.

### FIELD OF THE INVENTION

This invention relates to diagnostic aids for cystitis and, more specifically, to urinary trypsin inhibitors as a differential diagnostic aid for interstitial cystitis.

### BACKGROUND OF THE INVENTION

Interstitial Cystitis

Interstitial Cystitis (hereinafter “IC”) or chronic urinary inflammation is a chronic inflammatory condition involving the mucosa and muscle of the bladder. IC usually involves a damaged urothelium, or bladder lining. The cause of this urinary bladder disease is unknown (autoimmune, neurologic, allergic and genetic), but is characterized by pain associated with urination (dysuria), urinary frequency (as often as every 10 minutes), urgency, and pressure in the bladder and/or pelvis. Although there is a high incidence of IC in the population and 50% of all urinary tract infection work-ups are eventually diagnosed as cystitis, there is currently no good way to diagnose IC.

When a patient presents with an abnormal urge to urinate in combination with pain or a burning sensation, the clinician will first attempt to rule out an infection. In the absence of bacteriuria and pyuria, the condition is referred to a specialist and is generically labeled a Urologic Chronic Pelvic Pain Syndrome (UCPPS). CRP, CBC, and ESR tests can be used to rule out chronic infection and have a 3-7 day turn around at the primary care. Presently, a diagnosis of Cystitis can be confirmed only with a hydrodistention during cystoscopy with biopsy. X-Ray or ultrasound check look for abnormalities in the kidneys. Anatomic abnormalities may need to be surgically treated. Surgical interventions are rarely used for cystitis. Treatments include elimination diet, astringent instillations (clorpactin or silver nitrate), rescue instillation (elmiron or heparin, cystistat, lidocaine and sodium bicarbonate), transcutaneous electrical nerve stimulation (TENS), and antidepressant to fight neuroinflammation.

There is a need for a fast, noninvasive, high sensitivity, high specificity point-of-care differential diagnostic for cystitis.

### SUMMARY OF THE INVENTION

The invention in one aspect includes correlating the concentration of at least one urinary trypsin inhibitor with a diagnosis of interstitial cystitis. In a preferred embodiment, a rule out of an infection will be used in conjunction with the at least one urinary trypsin inhibitor test for a more specific result.

The invention in a second aspect is a new and improved test device to aid in the diagnosis of interstitial cystitis including a support structure, at least one test for infection disposed on the support structure, and at least one test for at least one urinary trypsin inhibitor disposed on the support structure.

The invention in another aspect includes a method aiding in the diagnosis of interstitial cystitis, including the steps of determining that a test result of the patient sample is positive for at least one urinary trypsin inhibitor; and reporting data indicative of a likelihood that the patient has interstitial cystitis. In a preferred embodiment, the method further includes determining that a test result of a patient sample is negative for infection.

The invention in yet another aspect includes an analyzer useful in the diagnosis of interstitial cystitis structured and arranged to read and determine results of at least one test for at least one urinary trypsin inhibitor. In a preferred embodiment, the analyzer is structured and arranged to read and determine results of at least one test for infection. The read results are stored by the analyzer as test result data. A processor for operating the analyzer executes software to correlate the test result data of the at least one test for infection (in the preferred embodiment) and the at least one test for at least one urinary trypsin inhibitor with interstitial cystitis. The correlation includes creating output data indicative of a likelihood of interstitial cystitis if the test result data for the at least one test for infection is negative (in the preferred embodiment) and the at least one test for at least one urinary trypsin inhibitor is positive.

Another aspect of the invention includes a kit for aiding in the diagnosis of interstitial cystitis. The kit includes a test for an infection marker and a test for at least one urinary trypsin inhibitor. Preferably, the kit contains instructions as to how to correlate the test results with interstitial cystitis.

Additional features and advantages of the invention will be made apparent from the following detailed description of illustrative embodiments that proceeds with reference to the accompanying drawings.

### BRIEF DESCRIPTION OF THE DRAWINGS

A more complete appreciation of the present invention and many of the attendant advantages thereof will be readily understood by reference to the following detailed description when taken in conjunction with the accompanying drawings, in which:

FIG. 1 is a schematic of the physiological process behind inflammation.

FIG. 2 illustrates an exemplary embodiment of the present invention in the form of a urine strip.

FIG. 3 is an exemplary schematic of an analyzer according to the present invention.

FIG. 4 depicts an exemplary process according to the present invention

### DESCRIPTION OF THE PREFERRED EMBODIMENTS

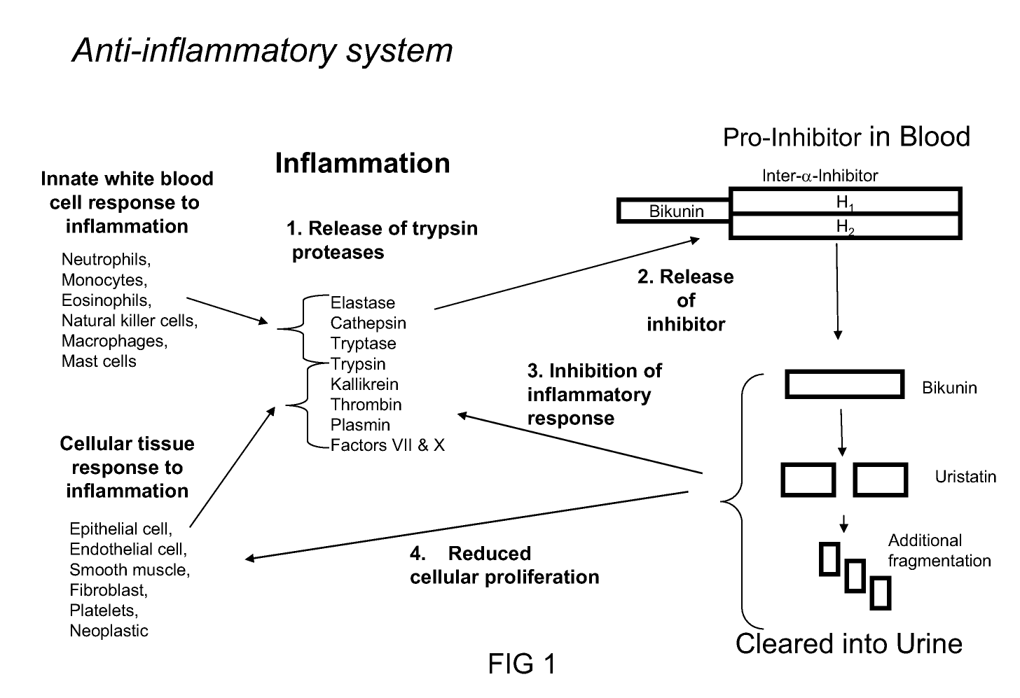

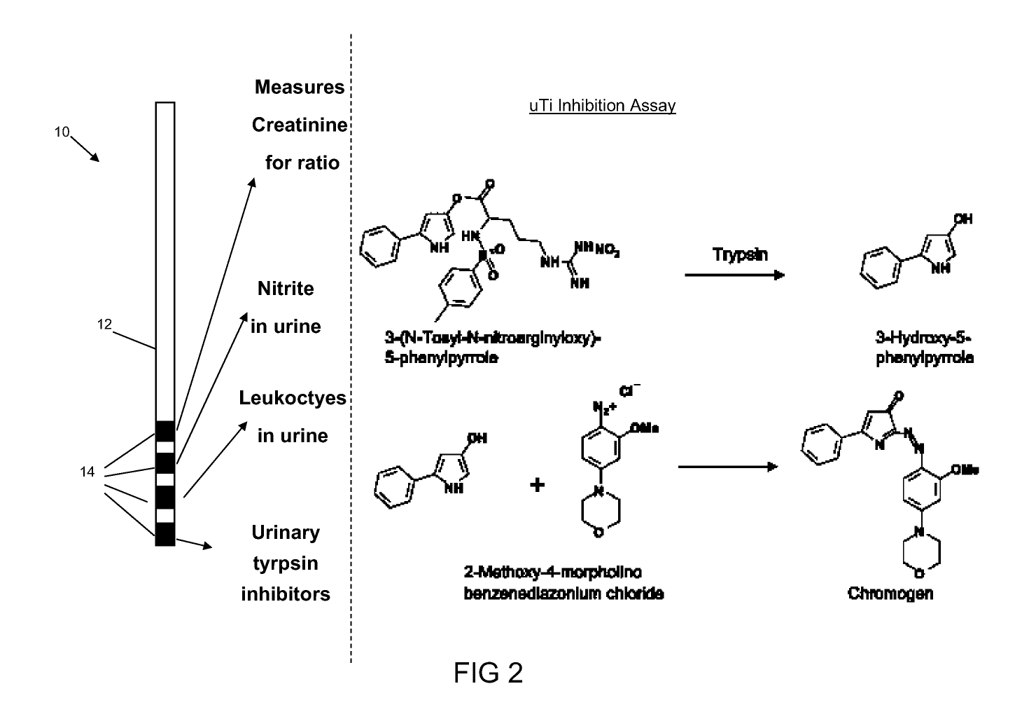

Referring now to the drawings in which like reference characters designate identical or corresponding parts throughout the several views, a preferred embodiment of the invention will now be described with reference to FIGS. 1-4.

It is to be understood that the invention described herein is not limited to particular methods, reagents, devices, systems, compounds, compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise.

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The invention employs biological fluids as the biological samples for use in the present invention. Preferably, the biological fluid is urine or blood, but other biological fluids can be used. Methods of obtaining biological samples from subjects are known in the art and are not described herein in detail.

Except when noted, the terms “subject” or “patient” are used interchangeably and refer to mammals such as human patients and non-human primates, as well as experimental animals such as rabbits, rats, and mice, and other animals.

The present invention includes the discovery that IC is correlated with, and can therefore be diagnosed with clinical specificity by detection of, an above normal presence of Urinary Trypsin Inhibitors in a patient's urine sample and, in a preferred embodiment, an absence of one or more markers correlated with infection. New and improved devices, methods, systems, and kits based on this novel concept are described herein. Prior to elaborating on the specific embodiments of the invention, it is important to have an understanding of Urinary Trypsin Inhibitors and their role in infection and the inflammatory processes.

Urinary Trypsin Inhibitors (Hereinafter “uTi's”)

uTi's inhibit one or more of the Serine proteases. Trypsin is a member of the family of Serine proteases, i.e. enzymes, that includes trypsin, elastase, kallikrein, plasmin, thrombin, chymotrypsin, and cathepsin, among others. This group of inhibitors primarily forms after an increase in the number of white blood cells in the body due to the release of elastase during infection or inflammation. uTi's are not normally found in the urine produced by healthy individuals. The amount is elevated in those whose bodies have bacterial infections and inflammatory disorders or other maladies such as malignant tumors, kidney disease, myocardial infarction, lung emphysema, surgical trauma, and kidney stones among others.

With reference now to FIG. 1, when infections and/or inflammation occur, the body's response involves the production of serine proteases, such as elastase, released by neutrophils. Non-inhibitor forms of uTi, called pro-inhibitors, such as interleukin-.alpha.-inhibitor (I-.alpha.-I) and the pre-interleukin-.alpha.-inhibitor (P-.alpha.-I), circulate freely in plasma of healthy and diseased individuals. Serine proteases cause proteolysis of the pro-inhibitors and release the lower molecular weight uTi's into active function. The released uTi's act on serine proteases and are later excreted in the urine. Discovered in 1909, urine trypsin inhibitors are Kunitz-type protease inhibitors and have been named HI-30, Mingin, Urinastatin, Serpin, and Ulinastatin over the years with the scientific community settling on the name Bikunin for a prevalent fragment of about 30 Kda molecular weight. The amino acid sequence of the Bikunin inhibitor fragment is known. It contains two Kunitz inhibitory binding domains and a large and variable chondroitin sulfate chain. See the International Journal of Biochemistry and Cell Biology 32 (2000) 125-137 incorporated herein by reference.

The amount of uTi has been measured by several methods, e.g. enzyme inhibition, antibody stains, latex agglutination, and radioimmunoassay. During the cross-reactivity studies of a polyclonal antibody for uTi, a discovery was made that further breakdown of the inhibitory Bikunin occurs during the acute phase infections in patients leading to the formation of other inhibitory uTi's containing both Kunitz inhibitor domains, but lacking the chondroitin sulfate chain. This uTi, termed Uristatin, has a molecular weight of about 17 kDa. In a paper incorporated herein by reference (Clinical Chemistry Acta (2004) 341, 73-81) reporting tests with a dipstick for detecting urinary trypsin inhibitors, Pugia et al showed that the dipstick reported the presence of two forms for uTi's; Bikunins and Uristatins. They identified the typical molecular weight of Bikunin (30.9 kDa) and three key forms of the Uristatin, designated Uristatin-1 (5.9 kDa) with a range of from about 2 to about 9 kDa for fragments and variants., Uristatin-2 (8.5 kDa) with a range of from about 2 to about 12 kDa for fragments and variants. The combination of Uristatin-1 and Uristatin-2 was termed Uristatin (17.4 kDa). Uristatin's molecular weight of about 17 kDa has a range of from about 11 to about 22 kDa for fragments and variants. It is now known that the presence of inflammation leads to inter-α-inhibitors breakdown and forms bikunin and uristatin. Cleavage of the chondoroitin sulfate chain from bikunin produces uristatin.

Uristatin-1 and -2 refer to protein fragments of uristatin which contain either of the kunitz type inhibitor domains 1 or 2. All forms of Uristatin lack the chondroitin sulfate chain, and are very prevalent in patient specimens when analyzed by electrophoresis. Additionally, all uristatin forms are inhibitory to the trypsin family of proteases; therefore they contain either one of the two Kunitz inhibitory domains that inhibit the protease active site upon binding. Uristatin-1 contains binding domain 2 and Uristatin-2 contains binding domain 1. See the International Journal of Biochemistry and Cell Biology 32 (2000) 125-137 incorporated herein by reference.

It was further noted that, given the conditions of the patient used for collection, the typical molecular weights of uTi's could vary considerably. Additional variations of Bikunin and Uristatin are due to fragmentation of peptide structure, variations in the peptide sequence and variations in carbohydrate sequences attached to the Bikunin and Uristatin. Variations in molecular weight resulting from fragments occur by cleavage of the peptide sequence. A high degree of fragmentation is expected during inflammation as the inhibitors are exposed to the proteases that can cause cleavage. Elongation and fragmentation of the carbohydrate portions was also expected during inflammation as the glycoprotein are metabolized by a number of glycosyl transferases and glycosidases causing a great number of possible variants to the chondroitin sulfate chain attached to Bikunin and to the sugar side chains attached to Uristatin. Additional variations also occur by aggregation of the fragments into diners or higher oligomers, especially through association and metabolism of the carbohydrate portions. Therefore the functional uTi proteins represent a range of possible proteins around a typical molecular weight.

uTi's are key anti-inflammatory proteins that slow down immune cell damage of tissues. Trypsin Serine proteases released by white blood cells and tissues. Bikunin and uristatin are different complex glycoconjugated forms. Free Bikunin and uristatin inhibitor levels in blood are very low since more than 99% are cleared relatively quickly into urine. Most importantly, it was discovered that Bikunin and uristatin are only in urine during inflammation due to infections or continued inflammatory responses.

Referring again to FIG. 1, the steps of the inflammation process are shown

and described: Step 1: Serine proteases of the trypsin family (Elastase, Cathepsin, Tryptase, Trypsin, Kallikrein, Thrombin, Plasmin and Factors VII & X) are increased during inflammation by innate immune cells (e.g. White Blood Cells; Neutrophils, Monocytes, Eosinophils, Natural killer cells, Macrophages, Mast cells) and affected tissues (Epithelial, Endothelial, Smooth muscle, Fibroblast, Platelets and Neoplastic). Step 2: These proteases produce inflammatory response of vascular dilation, coagulation, and Leukocyte infiltration with destruction of phatogens. Step 3: Serine proteases (mainly elastase) liberate plasma bikunin (Bik) from inter-α-inhibitor (I-α-I), activating the inhibitory response. Bik is rapidly excreted into urine. Step 4: Bik inhibit serine proteases as an anti inflammatory response controlling remodeling, smooth muscle contraction, fluid/electrolyte balance and protecting tissue from Leukocyte damage. Step 5: Bik signals cells to reduce proliferation, inflammation cytokine mediator release, growth factor activation, uPA activation and intra-cellular Ca+. Step 6: Bik is rapidly metabolized and excreted into urine. Uristatin (Uri) is a primary form in urine and lacks the O-linked glycoside.

Trypsin release and inhibitor levels were studied in cell culture work with p kidney proximal tubular epithielal cells, kidney mesangial cells, muscle myoctye and cancer cell cultures where cell extracts were measured by enzyme substrates, ELISA and western blot immunoassays. Bik levels were <=0.1 mg/L for all cell extracts measured by ELISA and western blot immunoassays showing that Bik is not normally expressed. Serine protease activity were measured using trypsin specific chromogenic substrate calibrated to trypsin standards. Serine protease inhibitors were measured by the inhibition of trypsin substrate hydrolysis. Cells were cultured, synchronized, lyzed and homogenates centrifuged with the resulting supernatants (Sup) and pellet residues (Pel) suspended in buffer and stored at −20° C. until tested. The results showing that serine protease are 8 to 70 times high than serine proteases under normal condition. Kidney tissue cells produced more serine protease than muscle or cancer cells. During inflammation the pathological urine levels of Bik are enough to inhibit all cellular trypsin activity showing that serine inhibitors to be 2 to 10 times higher than serine protease inhibitors.

A number of significant behaviors have been identified by the present inventor with regard to uTi's. Testing has shown that bik/uristatin is only present in inflammation due to acute bacteria infection or chronic urinary tissue inflammation (Interstitial cystitis). Significantly, bik/uristatin is not elevated by viral infections because of the differences in the immune response to viruses. Furthermore, uTi levels were found to be unaffected by the following conditions:

Cardiovascular disease: Hypertension, lipidemia, ACS, atheoscloresis, & chest pain.

Common cold: streptococcal (strep throat), laryngitis, sinus congestion sinusitis & whooping cough

Non-systemic infections: foot ulcers, skin infection, toe infection, ankle infection & ear infection.

Non-urinary tract inflammation: cardiovascular inflammation, allergy, allergic reaction, asthma.

Complex regional pain syndrome: fibromyalgia.reaction, RSD.

Acute trauma: Appendicitis, pancreatitis, trauma and surgical trauma

Cancer: colon cancer, breast cancer & benign tumor.

Neuoropathies: fibromyalgia and migranes.

Osteoarthritis: osteopenia and osteporosis.

Metabolic: Hypothyroid, hyperthyroid, gout, anemia, diabetes & obesity.

Miscellaneous: GERD, DAU, tuberculosis, otitis media & cellulitis.

Medications: Nitroglycerine, anti-inflammatory, anti-hypertensive, lipid reducing & anti-thrombotic.

These findings led to the discovery that uTi's could be used as a differential diagnostic for IC.

Tests and Test Methods

The term “test” as used herein can be construed as any method of detecting and measuring the presence and concentration of uTi's and the infection markers of the present invention. Both quantitative and semi-quantitative measurements are included. It is further understood that many test formats can be used in the present invention. A variety of assays for detecting uTi's and the infection markers are well known in the art and include, for example, enzyme inhibition assays, antibody stains, latex agglutination, and immunoassays, e.g., radioimmunoassay.

Immunoassays that determine the amount of a protein in a biological sample typically involve the development of antibodies against the protein. The term “antibody” herein is used in the broadest sense and refers to, for example, intact monoclonal antibodies, polyclonal antibodies, multispecific antibodies (e.g., bispecific antibodies), and to antibody fragments that exhibit the desired biological activity (e.g., antigen-binding). The antibody can be of any type or class (e.g., IgG, IgE, IgM, IgD, and IgA) or sub-class (e.g., IgG1, IgG2, IgG3, IgG4, IgA1 and IgA2).

Immunoassays that determine the amount of uTi's in a biological sample typically involve the development of antibodies against the uTi being measured. Polyclonal antibodies produced from rabbits inoculated with purified bikunin are cross-reactive with inter-α-inhibitor and are not specific to the various forms of bikunin and uristatin fragments. Monoclonal antibodies could potentially separate the various forms of bikunin and detect uristatin lacking glycoconjugation. Antibodies for detecting bikunin and uristatin are known in the art, for example a monoclonal antibody secreted by one of the group of hybridomas ATCC 421-5GX.1A8.5C1, ATCC 420-5D11.5GX.1E4, and ATCC 421-3G5.4C5.3B6, see also for example, Pugia et al., 2007, Glycoconj J 24; 5-15 and co-owned U.S. Publication 20070020683, as well as U.S. Pat. No. 6,995,921 all incorporated herein by reference in their entirety for any and all purposes. Similarly, antibodies for detecting infection markers are also well know to those skilled in the art and will not be detailed herein.

Immunoassays, including radioimmmunoassays and enzyme-linked immunoassays, are useful in the methods of the present invention. A variety of immunoassay formats, including, for example, competitive and non-competitive immunoassay formats, antigen capture assays and two-antibody sandwich assays can be used in the methods of the invention (Self and Cook, Curr. Opin. Biotechnol. 7:60-65 (1996)). In an antigen capture assay, antibody is bound to a solid phase, and sample is added such that the analyte, e.g., bikunin or an infection marker, is bound by the antibody. After unbound proteins are removed by washing, the amount of bound analyte can be quantitated, if desired, using, for example, a radioassay (Harlow and Lane, Antibodies A Laboratory Manual Cold Spring Harbor Laboratory New York, 1988)) Immunoassays can be performed under conditions of antibody excess, or as antigen competitions, to quantitate the amount of analyte and, thus, determine a level of uTi and/or infection marker.

Urinalysis strip methods, such as reagent strip methods can be used for the rapid detection of trypsin inhibitors in urine (Pugia et al., Clin Chim Acta, 2004; 341:73-81, incorporated herein by reference in its entirety and for all purposes). Referring to FIG. 1, an exemplary urine strip or dipstick 10 is shown. The strip has a nonabsorbent handle 12 which supports absorbent pads 14 containing reagents for the various tests. In this embodiment, the strip contains reagent pads for creatinine, leukocyte, nitrite, and a uTi. It is understood that pads for other tests may also be disposed on the strip.

Immunochromatographic strip formats have become increasingly popular for qualitative and semi-quantitative assays which use visual detection schemes. This type of immunoassay involves the application of a liquid test sample suspected of containing an analyte to be detected to an application zone of a lateral flow immunochromatographic test strip. The strip is comprised of a matrix material through which the test fluid and analyte suspended or dissolved therein can flow by capillarity from the application zone to a capture zone where a detectable signal, or the absence of such, reveals the presence of the analyte. Typically, the strip will include means for immunospecifically binding the analyte to be detected with its specific binding partner which bears the detectable label. Alternative arrangements are also well known in the art.

Enzyme-linked immunosorbent assays (ELISAs) can be used in the present invention. In the case of an enzyme immunoassay, an enzyme is typically conjugated to the second antibody, generally by means of glutaraldehyde or periodate. As will be readily recognized, however, a wide variety of different conjugation techniques exist which are readily available to one skilled in the art. Commonly used enzymes include, for example, horseradish peroxidase, glucose oxidase, β-galactosidase and alkaline phosphatase, amongst others. The substrates to be used with the specific enzymes are generally chosen for the production, upon hydrolysis by the corresponding enzyme, of a detectable color change. It is also possible to employ fluorogenic substrates, for example, which yield a fluorescent product. An enzyme such as horseradish peroxidase (HRP), alkaline phosphatase (AP), β-galactosidaseor urease can be linked, for example, to an anti-bikunin antibody or to a secondary antibody for use in a method of the invention. A horseradish-peroxidase detection system can be used, for example, with the chromogenic substrate tetramethylbenzidine (TMB), which yields a soluble product in the presence of hydrogen peroxide that is detectable at 450 nm. Other convenient enzyme-linked systems include, for example, the alkaline phosphatase detection system, which can be used, for example, with the chromogenic substrate p-nitrophenyl phosphate to yield a soluble product readily detectable at 405 nm. Similarly, a β-galactosidase detection system can be used with, for example, the chromogenic substrate o-nitrophenyl-β-D-galactopyranoside (ONPG) to yield a soluble product detectable at 410 nm, or a urease detection system can be used with, for example, a substrate such as urea-bromocresol purple (Sigma Immunochemicals, St. Louis, Mo.). Useful enzyme-linked primary and secondary antibodies can be obtained from a number of commercial sources such as Jackson Immuno-Research (West Grove, Pa.).

In certain embodiments, uTi and/or the infection maker can be detected and measured using chemiluminescent detection. For example, in certain embodiments, bikunin specific antibodies are used to capture bikunin present in the biological sample and an antibody specific for the specific antibodies and labeled with an chemiluminescent label is used to detect the bikunin present in the sample. Any chemiluminescent label and detection system can be used in the present methods. Chemiluminescent secondary antibodies can be obtained commercially from various sources such as Amersham. Methods of detecting chemiluminescent secondary antibodies are known in the art and are not discussed herein in detail.

Fluorescent detection also can be useful for detecting uTi and/or the infection maker of the present invention. Useful fluorochromes include, for example, DAPI, fluorescein, lanthanide metals, Hoechst 33258, R-phycocyanin, B-phycoerythrin, R-phycoerythrin, rhodamine, Texas red and lissamine Fluorescent compounds, can be chemically coupled to antibodies without altering their binding capacity. When activated by illumination with light of a particular wavelength, the fluorochrome-labelled antibody adsorbs the light energy, inducing a state of excitability in the molecule, followed by emission of the light at a characteristic color visually detectable with a light microscope.

Radioimmunoassays (RIAs) can be useful in certain methods of the invention. Such assays are well known in the art. Radioimmunoassays can be performed, for example, with ¹²⁵I-labeled primary or secondary antibody (Harlow and Lane, supra, 1988).

A signal from a detectable reagent can be analyzed using any methods known in the art. A signal can be measured, for example, using a spectrophotometer to detect color from a chromogenic substrate; a radiation counter to detect radiation, such as a gamma counter for detection of ¹²⁵I; or a fluorometer to detect fluorescence in the presence of light of a certain wavelength. Where an enzyme-linked assay is used, quantitative analysis of the amount of the uTi or infection marker can be performed using a spectrophotometer such as an EMAX Microplate Reader (Molecular Devices; Menlo Park, Calif.) in accordance with the manufacturer's instructions. The assays of the invention can be automated or performed robotically, if desired, and the signal from multiple samples can be detected simultaneously.

The methods of the invention also encompass the use of capillary electrophoresis based immunoassays (CEIA), which can be automated, if desired. Immunoassays also can be used in conjunction with laser-induced fluorescence as described, for example, in Schmalzing and Nashabeh, Electrophoresis 18:2184-93 (1997), and Bao, J. Chromatogr. B. Biomed. Sci. 699:463-80 (1997).

Sandwich enzyme immunoassays also can be useful in certain methods of the invention. In a two-antibody sandwich assay, a first antibody is bound to a solid support, and the antigen is allowed to bind to the first antibody. The amount of the analyte can be quantitated by measuring the amount of a second antibody that binds to it.

Quantitative western blotting also can be used to determine a level of uTi and/or the infection maker in the present methods. Levels of uTi and/or infection marker can also be determined using protein microarrays. Methods of producing protein microarrays that can be adapted for detecting levels of protein in a clinical sample are well known in the art.

In certain embodiments, a sample is analyzed by means of a microfluidic device. Microfluidic devices generally comprise solid substrates containing an arrangement of capillaries and wells through which the samples and the reagents flow to mix, react, or to be read. Examples of microfluidic devices can be found in U.S. Pat. Nos. 7,094,354; 7,125,711; 7,347,617; and 7,435,381 incorporated herein by reference in their entirety and for all purposes.

In certain embodiments, a sample is analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

Protein biochips are biochips adapted for the capture of peptides. Many protein biochips are described in the art. These include, for example, protein biochips produced by Ciphergen Biosystems, Inc. (Fremont, Calif.), Packard BioScience Company (Meriden Conn.), Zyomyx (Hayward, Calif.), Phylos (Lexington, Mass.) and Biacore (Uppsala, Sweden). Examples of such protein biochips are described in the following patents or published patent applications: U.S. Pat. No. 6,225,047; PCT International Publication No. WO 99/51773; U.S. Pat. No. 6,329,209, PCT International Publication No. WO 00/56934 and U.S. Pat. No. 5,242,828, incorporated herein by reference in their entirety and for all purposes.

For use herein, the assay methods can involve capturing the uTi and/or the infection marker onto a solid substrate. Typically they will be captured using a biospecific capture reagent such as an antibody and, in particular, an antibody used in an immunoassay. Biospecific capture reagents include those molecules that bind a target analyte with an affinity of, for example, at least 10⁻⁹ M, 10⁻¹⁰ M, 10⁻¹¹ M or 10⁻¹² M. These molecules also can be captured with non-specific methods, such as chromatographic materials.

In certain embodiments of the present invention, the uTi and/or the infection maker will be detected by mass spectrometry. Examples of mass spectrometers are time-of-flight, magnetic sector, quadrupole filter, ion trap, ion cyclotron resonance, electrostatic sector analyzer and hybrids of these.

A preferred mass spectrometric technique for use in the invention is “Surface Enhanced Laser Desorption and Ionization” or “SELDI,” as described, for example, in U.S. Pat. No. 5,719,060 and No. 6,225,047, both to Hutchens and Yip, each of which is incorporated herein by reference in its entirety and for all purposes. This refers to a method of desorption/ionization gas phase ion spectrometry (e.g., laser desorption/ionization mass spectrometry) in which an analyte is captured on the surface of a SELDI probe that engages the probe interface of the mass spectrometer.

When using enzyme inhibition to measure uTi concentration, colorimetric enzyme substrates have been used to measure the extent of the inhibition. The method has been recently adapted to automated measurement on clinical analyzers (S. Kuwajima, et al., loc. cit.). Such analytical techniques typically involve contacting the urine sample with a trypsin substrate attached to a chromophore at either arginine or lysine, because trypsin cleaves arginine and lysine. The concentration of UTI in the urine sample is inversely proportional to the intensity of the colored response of the chromophore since UTI inhibit trypsin activity according to their concentration in the fluid test sample. Several colorimetric and fluorogenic trypsin substrates are commercially available, including N.alpha.-benzoyl-L-arginine p-nitroanilide (BAPNA), N.alpha.-benzoyl-D,L-arginine .beta.-naphthylamide (BANA) and N.alpha.-benzoyl-L-arginine-7-amido-4-methylcournarin.

In certain embodiments, the uTi and/or the infection marker can also be measured using enzyme inhibition methods and/or kinetic enzymatic assays. One example of an enzyme inhibition method for measuring bikunin involves the addition to the biological sample of known amounts of trypsin and then measuring the degree to which the trypsin has been inhibited. A substrate is used that is capable of producing a detectable response when it is added to a biological sample and the substrate is cleaved by trypsin to yield detectable byproducts. If trypsin inhibitors are present, the response is reduced, because some of the available substrate is not cleaved. Methods of measuring bikunin using enzyme inhibitory assays are well known in the art. Known indicating trypsin substrates are aromatic amides of N.alpha.-protected arginine. When trypsin hydrolyzes these known substrates, the amide bond is cleaved and an aromatic amine is released. In the case of BAPNA, the amide bond is cleaved and yellow-colored p-nitroaniline is liberated and measured with a spectrophotometer. With BANA, 2-amino-naphthalene is produced, and it is detected by diazotization and coupling with N-(1-naphthyl)-ethylenediamine to form an azo dye (Goldberg, et al., Cancer 11, 283 (1958)). 7-Amino-4-methylcoumarin is released by hydrolysis of N.alpha.-benzoyl-L-arginine-7-amido-4-methylcoumarin, and this fluorescent product is measured with a fluorometer. These substrates are used for measuring trypsin activity in liquid-phase assays but are not well suited for use in dry-phase formats, such as dip-sticks, which are typically read visually or with simple reflectance instruments. Assays suitable for use in liquid and dry-phase assays have been developed, see for example, U.S. Pat. Nos. 6,955,921, 6,770,764, 7,001,737 and Pugia et al., 2002, Clinical Biochemistry, 32:105-11, each of which is incorporated herein by reference in its entirety and for all purposes.

In certain embodiments, the uTi can be measured by measuring the level of expression of the genes encoding for the uTi. Nucleic acid assays are known to those skilled in the art and include, for example, Southern analysis, Northern analysis, dot blots, S1 analysis, amplification techniques such as PCR, and in situ hybridization.

### EMBODIMENTS

A test device according to the present invention includes a test for one or more urinary trypsin inhibitors and a test for an infection marker. Infection markers may include, but are not limited to, urine tests for leukocyte, nitrite, pH, bacteria, casts and the like and blood tests, such as CBC, CRP, NGAL, elastase, cytokine, acute phase reactants, growth factors and the like.

Referring now to FIG. 1, an exemplary urine strip 10 is shown. The construction of urine strips are well known in the art. The strip has a nonabsorbent handle 12 which supports absorbent pads 14 containing reagents for the various tests. In this embodiment, the strip contains reagent pads for creatinine, leukocyte, nitrite, and a Urinary Trypsin Inhibitor. It is understood that pads for other tests may also be disposed on the strip. The color responses from the urine strip could be read by a reflectance spectroscope, such as the CLINITEK STATUS® analyzer available from Siemens Healthcare Diagnostics Inc. to detect color response. Reflectance spectroscopes for reading test results based on color change are well know in the art and a description of such an analyzer's operation is not necessary here. See U.S. Pat. No. 5,877,863 incorporated herein by reference in its entirety and for all purposes.

The uTi pad in a preferred embodiment includes an enzyme inhibition method and contains tyrpsin enzyme, tyrpsin enzyme substrate to produce color, buffers and stabilizing agents. Methods of manufacturing the dry-phase assay for uTi can be found in co-owned U.S. Pat Nos. 6,955,921 and 7,001,737 previously incorporated herein be reference in their entirety. The reagent pad is structured and arranged so that the absence of color can be measured by an analyzer and analyzed in accordance with a method of the present invention described below. It is important that the uTi reagent in the pad does not interfere with the reactions on the other reagent pads or vice versa. In the above urine strip example, it was determined through experimentation that the uTi reagent did not interfere with the leukocyte, nitrite, or creatinine reagents and vice versa.

A method according to a preferred embodiment of the present invention includes determining that a test result of a patient sample is negative for infection and determining that a test result of the patient sample is positive for above the normal threshold for Urinary Trypsin Inhibitor. As will be discussed in greater detail below, if the test for infection is negative and the test for inflammation is positive, there is a significant likelihood that the patient has interstitial cystitis and that result should be reported. The marker for infection can be any infection marker alone or in combination, including, but not limited to, leukocyte, nitrite, or the like, as described above. The marker may be detected in urine or blood. The Urinary Trypsin Inhibitor marker may be any of the Urinary Trypsin Inhibitors described above, alone or in combination. In a preferred embodiment, the test is indicative of the presence of bikunin, uristatin, and uristatin-1 and -2. The uTi test is preferably carried out by contacting a sample of a patient's urine with a reagent method for detecting a uTi. As an alternative to a reagent based on enzyme inhibition, is an immunoassay uTi using at least one antibody for uTi detailed above. Although Urinary Trypsin Inhibitors alone can be used as a marker for interstitial Cystitis, it has been found that there are fewer false positives when uTi are used in conjunction with a rule out of infection.

The method may also include determining that the patient does not have a condition that could cause a false positive for uTi, such as pregnancy. The method in some embodiments also includes determining that the patient does not have a condition that could cause a false negative uTi, such as HIV or AIDS. Additionally, the method may include determining that the patient does not have a condition that could cause a false negative in the infection test, such as leukemia.

In determining whether a test for a marker is positive or negative, the result of the test is compared to predetermined threshold levels. If the test result value is above the threshold, it is indicative of a higher than normal presence of the analyte being measured and the test may be considered positive for the presence of the analyte. If the result value is below the threshold then the analyte is not present in an amount that is above the norm and the test is negative for presence of the analyte. It is understood that certain tests may be positive if the expression or concentration of an analyte is below the threshold level. It is also common in semi-quantitative tests to establish a few threshold levels. The first being the norm. A second level being trace amounts of an analyte or a slightly above normal presence of an analyte. A third threshold can also be established indicative of a severe or high degree of analyte concentration. Obviously, the number of threshold levels established would depend upon the ability to measure and distinguish between the threshold levels and the clinical utility of being able to distinguish those levels.

The normal uTi threshold level for a patient without an infection or inflammation is less than about 6 mg of uTi per liter of urine Immunoassay for specific forms of uTi have lower thresholds. However, for clinical testing purposes it has been found that a threshold level of about 12.5 mg of uTi per liter of urine sufficiently distinguishes negative uTi from positive uTi and is readily distinguishable using optical analysis such as chromatography. If desired additional severity threshold levels can be established at about greater than 25 mg/L, but less than 50 mg/L, and another threshold level at greater than 50 mg/L of urine. It is understood that these threshold levels are examples and that other levels could be established. For clinical use it is important that the various thresholds can be distinguishable from one another in order to be relevant.

It is also possible to establish thresholds based on the ratio of uTi and creatinine. Creatinine is known to be an indicator of a dilute urine sample, see for example U.S. Pat. Nos. 6,306,660 and 6,436,721 incorporated herein by reference in their entirety for any purpose. In this case, the normal threshold is less than about 0.1 mg/g of creatinine in urine. However, for clinical testing purposes it has been found that a threshold level of greater than about 12.5 mg/g of creatinine is sufficient to return a positive value. Additional severity thresholds can be set at greater than about 25 mg/g of creatinine.

Table 1 shows the results of a study conducted to show the correlation between uTi and cystitis. The study included 6,288 patient samples 5,798 of which were taken from patients that did not have IC and 490 of which had IC. Using uTi strips such as those discussed above, IC was diagnosed with a sensitivity of 99.8% and a specificity of 91%. Of the 510 patient with out cystitis, but with a positive Uristatin strip, 159 were show to have an infection using a urine culture and 115 were shown to have a systemic infection by a complete blood count (CBC). The addition of these test improved test to a sensitivity of 99.8% and a specificity of 96%.

TABLE 1

| | Threshold | Cystitis | | |
|---|---|---|---|---|
| Strip uristatin | level (mg/L) | NO | YES | |
| | 0 | 5288 | 1 | 5289 |
| | 12.5 | 364 | 286 | 650 |
| | 25 | 78 | 110 | 188 |
| | 50 | 68 | 93 | 161 |
| | SUM | 5798 | 490 | 6288 |
| | | Uri strip | | |
| | Threshold | >12 mg/L | | |
| | Sensitivity | 99.8% | | |
| | Specificity | 91% | | |
| | False Negative | 1 | | |
| | False Positive | 510 | | |
| | True Positive | 489 | | |
| | True Negative | 5288 | | |
| | PPV | 49% | | |
| | NPV | 100% | | |

In contrast, previously known tests had significantly less sensitivity and specificity for diagnosing IC. See the results in Tables 2-4. Traditional markers for infection show a sensitivity and specificity for IC that is so low that, used alone, they provide no clinical significance for IC.

TABLE 2

| | | Cystitis | | |
|---|---|---|---|---|
| WBC/NIT | | NO | YES | |
| | neg | 5048 | 342 | 5390 |
| level | pos | 756 | 148 | 904 |
| | | | | 0 |
| | | | | 0 |
| | SUM | 5804 | 490 | 6294 |
| | | WBC and NIT | | |
| | threshold | trace and pos | | |
| | sens | 30.2% | | |
| | spec | 87% | | |
| | FN | 342 | | |
| | FP | 756 | | |
| | TP | 148 | | |
| | TN | 5048 | | |
| | PPV | 16% | | |
| | NPV | 94% | | |

TABLE 3

| | | Cystitis | | |
|---|---|---|---|---|
| CBC | | NO | YES | |
| | viral | 94 | 0 | 94 |
| level | neg | 5607 | 490 | 6097 |
| | bact | 103 | 0 | 103 |
| | | | | 0 |
| | SUM | 5804 | 490 | 6294 |
| | | CBC | | |
| | threshold | >12400 | | |
| | sens | 0.0% | | |
| | spec | 98% | | |
| | FN | 490 | | |
| | FP | 103 | | |
| | TP | 0 | | |
| | TN | 5701 | | |
| | PPV | 0% | | |
| | NPV | 92% | | |

TABLE 4

| | | Cystitis | | |
|---|---|---|---|---|
| CRP | | NO | YES | |
| | <=10 | 5655 | 489 | 6144 |
| level | >10 | 149 | 1 | 150 |
| | | 0 | 0 | 0 |
| | | | | 0 |
| | SUM | 5804 | 490 | 6294 |
| | | CRP | | |
| | threshold | positive | | |
| | sens | 0.2% | | |
| | spec | 97% | | |
| | FN | 489 | | |
| | FP | 149 | | |
| | TP | 1 | | |
| | TN | 5655 | | |
| | PPV | 1% | | |
| | NPV | 92% | | |

Similarly, a kit according to the present invention includes a test for one or more urinary trypsin inhibitors and can be combined with one or more test for an infection. The tests may be attached to one sampling device, for example if they are on the same urine strip, or may be separate test items. The items are preferably contained in appropriate packaging. Examples of infection tests include white blood cell measurements in urine or blood, cast measurements in urine, nitrite in urine, specific pathogen test in urine or blood. These additional methods are typically done by diagnostic chemistry, culture, molecular or immunoassay method. The kit preferably also includes a set of operator instructions including the method set forth above.

A device according to one embodiment of the present invention is a standard reflectance spectrometer that can read and measure color change from an assay, but in addition is programmed to read a uTi test and compare the results to one or more predetermined thresholds. It is also understood that the components of the analyzer, especially the processing and software components may be part of a unitary analyzer or part of a distributed system whereby the various components are operatively coupled by means of communication networks.

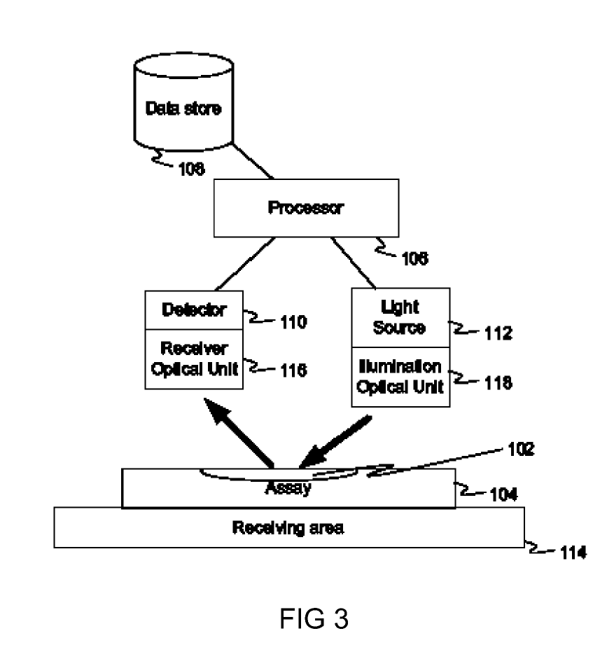

Referring now to FIG. 3, these analyzers generally include hardware for measuring color response of a test area 102 of reacted reagent on an assay 104. The system may include a processor 106 in connection with a datastore 108, a detector 110, and a light source 112. The system may include a receiving area 114 that secures the assay 104. The system may include a receiver optical unit 116 coupled with the detector 110. The system may include an illumination optical unit 118 coupled with the light source 112.

The assay 104 may be a card, strip, microfluidic chip, and/or immuno-chromatography device. The assay 104 may have a reagent. The reagent may react when in contact with a sample. The reaction may include a change in color. The color change may be indicative of the presence and/or absence of a composition within a sample and/or a characteristic of the sample. For example, the assay 104 may be a urinalysis assay 104. For example, the assay 104 may be a pH test.

The assay 104 may define a test area 102. The test area 102 may be the area of the assay 104 in contact with the sample. Light from the light source 112 and directed by the illumination optical unit 118 may reflect off of the surface of the assay 104 and/or the test area 102. The light reflected from the test area 102 of the assay 104 may correspond with the color response of the test area 102. The light reflected from the test area 102 may be within a field of view, as defined by the receiver optical unit 116 and/or the detector 110. The light reflected from the test area 102 may reach and/or be sensed by the detector 110. The detector 110 may measure the color and/or intensity of the light received.

The processor 106 may be any system, subsystem, and or component suitable for processing data and/or controlling the detector 110 and/or the light source 112. The processor 106 may be a microprocessor, a microcontroller, a collection of logical hardware components, and the like. The processor 106 may direct the light source 112 to illuminate. The processor 106 may direct the detector 110 to sense light. The processor 106 may receive a reading from the detector 110 corresponding to the light sensed by the detector 110. The processor 106 may be in connection with the datastore 108. The processor 106 may store readings received from the detector 110 at the datastore 108. The processor 106 may receive computer executable introductions from the datastore 108. Additionally, the computer readable instructions may be hardwired into the hardware. The computer executable instructions may direct the processor 106 to operate and/or control the detector 110 and/or the light source 112. Additionally, the computer executable instruction may direct the processor to compute and correlate test result readings with predetermined threshold levels stored in the datastore 108.

The light source 112 may be any system, subsystem, and or component suitable for generating light. For example, the light source 112 may be a light emitting diode (LED). Also for example, the light source 112 may be an incandescent light, fluorescent light, halogen light, and the like. The light source 112 may be an array of LEDS. The light source 112 may be controlled by the processor 106. The light source 112 may receive instructions from the processor 106 to illuminate according to a timing defined by the processor 106.

The light source 112 may be coupled with the illumination optical unit 118. The illumination optically unit may be any system, subsystem, and/or device suitable for directing in light from the light source 112 to the test area 102 and/or surface of the assay 104. The illumination optical unit 118 may provide a substantially uniform distribution of light from the light source 112 across the assay 104 in and/or around the test area 102. For example, the illumination optical unit 118 may be a light guide, a lightbox, an optical fiber, a conventional lens, a total internal reflection lens, and the like. For example, the optical unit may be a light guide with a circular cross-section, and/or a rectangular cross-section.

The detector 110 may be any system, subsystem, and/or component suitable for detecting light. The detector 110 may detect and/or sense the magnitude of light. For example, the detector 110 may return a result corresponding to the intensity of the light sensed by the detector 110. In an embodiment, the detector 110 may be a photo diode. In an embodiment, the detector 110 may be a charge coupled device (CCD) imager. The detector 110 may return a result corresponding to a color value associated with the light. For example, the detector 110 may return a result corresponding with the wavelength of light sensed by the detector 110. In an embodiment, the detector 110 may detect a luminance value associated with the magnitude of the intensity of the light sensed by the detector 110. In an embodiment the system may determine a reading for a plurality of wavelengths by directing the light source 112 to illuminate the plurality of wavelengths. The detector 110 may sense a luminance value associated with the respective wavelength. The processor 106 may coordinate the sequence of wavelengths illuminated by the light source 112 and/or the corresponding sequence of readings received from the detector 110.

The detector 110 may be coupled with a receiver optical unit 116. The receiver optical unit 116 in combination with the detector 110 may define a field of view. The field of view may define the scope of light that reaches the surface of the detector 110 and/or be sensed by the detector 110. The receiver optical unit 116 may include an aperture. The aperture may limit the amount of light that may reach the detector 110. The receiver optical unit 116 may be a light guide, an optical fiber, an axicon, an imaging lens, and the like.

The assay 104 may be secured by a receiving area 114. The receiving area 114 may hold the assay 104 in a position relative to the other components of the system. For example, the receiving area 114 may hold the assay 104 in a position relative to the light source 112 and/or the illumination optical unit 118. Also for example, the receiving area 114 may hold the assay 104 in a position relative to the detector 110 and/or the receiver optical unit 116. In an embodiment, the receiving area 114 may hold the assay 104 such that the test area 102 is co-linear with the detector 110 and the receiver optical unit 116.

In an embodiment, the light source 112 may include an array of light emitting diodes having an area of about 0.44 mm by 0.51 mm (+/−0.2 mm) and/or the area equivalent. The optical unit may include a light guide having a cross sectional area of about 2.7 mm by 2.7 mm (+/−0.5 mm) and/or the area equivalent. The head area for test area 102 may be substantially circular with a diameter of 1 mm (+/−0.2 mm) and/or the area equivalent.

In an embodiment, the system may include a second detector and a second lens. The second lens and the second detector may define a second field of view. The assay 104 may have a second test area that circumscribes the second field of view. The processor 106 may direct substantially simultaneous readings from the detector 110 and the second detector.

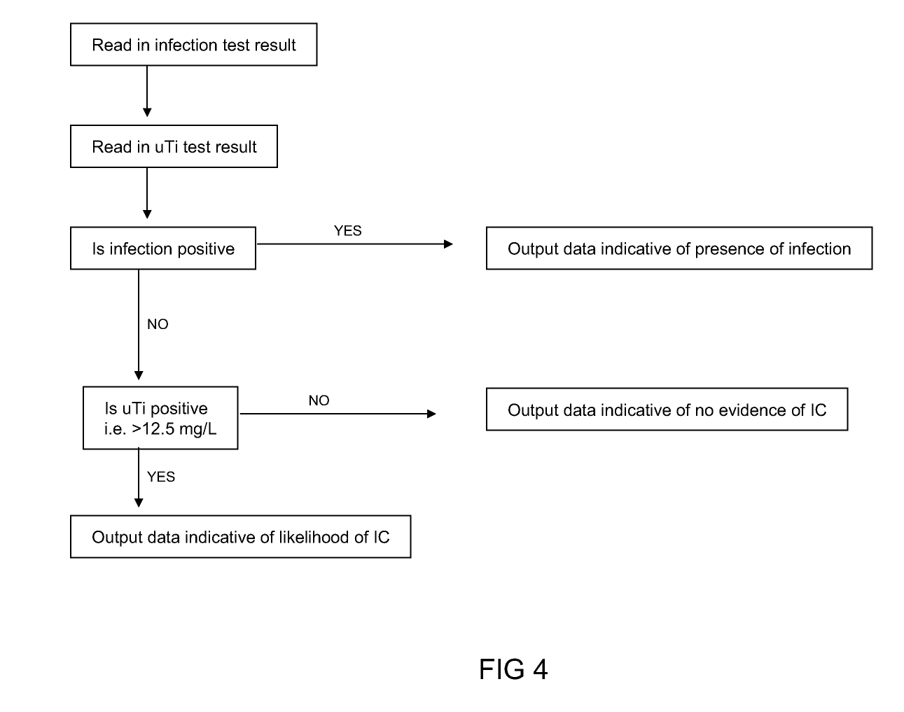

As mentioned above, the processor may execute computer readable instructions to operate the analyzer and to process the data resulting from the reading of the assay. Referring now to FIG. 4, a schematic showing the steps of the specially programmed computer readable instructions are shown. The assay for both the infection marker and the uTi marker are read by the detector and stored as test results in the datastore by the processor. The test results are then accessed from the datastore by the processor and compared against predetermined threshold values stored in the datastore. The threshold values have been discussed in detail above. If the comparison of the infection marker against the threshold for infection yields a positive result, then the processor is instructed to create an output indicative of the presence of infection. If the comparison yields a negative result against the threshold, then the uTi results are compared against the predetermined threshold values. If the uTi comparison yields a positive result, then the processor is instructed to create output data indicative of a likelihood of IC. If the uTi comparison yields a negative result, then the processor is instructed to create output data indicative of the lack of clinical evidence for IC. The actual quantitative result of the tests may also be included in or with the output data.

Output data can be a message reported to the operator of a device on the screen of an instrument or reported in conjunction with the test results. It can be reported to an LIS or HIS system electronically. The data can be in the form of a flag or warning message or any other indicator that can convey the desired output message. The output data is useful in diagnosing or ruling out IC and is intended to be used by a clinician as an aid in diagnosing IC and to tailoring the treatment of the patient.

While the present invention has been described in connection with the exemplary embodiments of the various figures, it is not limited thereto and it is to be understood that other similar embodiments may be used or modifications and additions may be made to the described embodiments for performing the same function of the present invention without deviating therefrom. Therefore, the present invention should not be limited to any single embodiment, but rather should be construed in breadth and scope in accordance with the appended claims. Also, the appended claims should be construed to include other variants and embodiments of the invention, which may be made by those skilled in the art without departing from the true spirit and scope of the present invention.

I claim:

1. A test device for interstitial cystitis comprising:
a support structure;
at least one test for infection disposed on said support structure; and
at least one test for at least one urinary trypsin inhibitor disposed on said support structure.

2. The test device of claim 1, wherein said at least one test for infection includes an absorbent pad containing a reagent that can indicate whether an analyte selected from the group consisting of leukocyte, nitrite, pH, bacteria, and casts in a urine sample is above or below a predetermined threshold level.

3. The test device of claim 1, wherein said at least one test for at least one urinary trypsin inhibitor includes an absorbent pad containing a reagent that can indicate whether at least one urinary trypsin inhibitor in a urine sample is above a predetermined threshold.

4. The test device of claim 3, wherein said at least one urinary trypsin inhibitor includes bikunin.

5. The test device of claim 3, wherein said at least one urinary trypsin inhibitor includes uristatin.

6. The test device of claim 1, wherein said support structure is a microfluidic chip.

7. The test device of claim 1, wherein said at least one test for infection and said at least one test for inflammation are disposed on the same or separate lateral flow device.

8. A method aiding in the diagnosis of interstitial cystitis, comprising the steps of:
measuring at least one urinary trypsin inhibitor in a patient sample;
reporting a likelihood that said patient has interstitial cystitis if said at least one urinary trypsin inhibitor is measured to be above a predetermined threshold.

9. The method of claim 8, further comprising the step of:
measuring at least one infection marker; and
wherein said step of reporting is only carried out if said at least one infection marker is below a predetermined threshold and said urinary trypsin inhibitor is measured to be above a predetermined threshold.

10. The method of claim 9, wherein said at least one infection marker is selected from the group consisting of leukocyte, nitrite, pH, bacteria, urine casts, CBC, CRP, NGAL, elastase, cytokine, acute phase reactants, and growth factors.

11. The method of claim 9, wherein said at least one urinary trypsin inhibitor includes bikunin.

12. The method of claim 9, wherein said at least one urinary trypsin inhibitor includes uristatin.

13. A system useful in the diagnosis of interstitial cystitis, comprising:
an analyzer structured and arranged to read results of at least one test for infection and at least one test for at least one urinary trypsin inhibitor and store said read results as test result data;
a processor for running software; and
said software, operable by said processor,
wherein said software operates to correlate said test result data of said at least one test for infection and said at least one test for at least one urinary trypsin inhibitor with interstitial cystitis;
wherein said correlation includes creating and outputting data indicative of a likelihood of interstitial cystitis if said test result data for said at least one test for infection is negative and said at least one test for at least one urinary trypsin inhibitor is positive.

14. The system of claim 13, wherein said software further comprises the functionality of creating and outputting data indicative of a lack of correlation between test results and interstitial cystitis if said at least one test for said at least one urinary trypsin inhibitor is negative.

15. The system of claim 13, wherein said software further comprises the functionality of outputting data in the form of a report or other human readable format.

16. The system of claim 13, wherein said at least one test for at least one urinary trypsin inhibitor includes a urine test for uristatin.

17. The system of claim 13, wherein said at least one test for at least one urinary trypsin inhibitor includes a urine test for bikunin.

18. The system of claim 13, wherein said processor and software are onboard said analyzer.

19. The system of claim 13, wherein said processor and software are remote from said analyzer.

20. A kit, comprising:
a urine test for infection;
a urine test for at least one urinary trypsin inhibitor; and
instructions for use including the method of claim 9.
